(19) United States
(12) Patent Application Publication
(10) Pub. No.: US 2021/0275771 A1
(43) Pub. Date: Sep. 9, 2021

(54) APPARATUS AND METHODS FOR CLEANING AND OXYGEN-ENRICHING AIR

(71) Applicant: OXIGEAR CORPORATION, Ontario (CA)

(72) Inventors: SIMON HO, Ontario (CA); BENNY HUM, Ontario (CA); DONNA SHUKARIS WALKER, Ontario (CA); ALEX SOCHANIWSKYJ, Ontario (CA); HOSSEIN KAZEMAIAN, Ontario (CA)

(21) Appl. No.: 16/316,339

(22) PCT Filed: Jul. 7, 2017
(86) PCT No.: PCT/CA2017/000168

§ 371 Date: Jan. 8, 2019

Related U.S. Application Data
(60) Provisional application No. 62/359,953, filed on Jul. 8, 2016.

(51) Int. Cl. A61M 16/22; A61M 16/00; A61M 16/10; A61M 16/20; B01J 20/28; B01J 20/18

(52) U.S. Cl. CPC A61M 16/22; A61M 16/0063; A61M 16/0093; A61M 16/101; A61M 16/107; A61M 2205/75; B01J 20/28004; B01J 20/18; B01J 20/28016; A61M 2205/584; A61M 16/208

(57) ABSTRACT

A portable breathing apparatus for oxygen enrichment of breathable air comprises: an adsorption vessel; an air compressor for pumping air into the adsorption vessel; a valve for purging pressure from the adsorption vessel; an adsorbent disposed within the adsorption vessel adsorbing a non-oxygen constituent of air when the vessel is pressurized, thereby producing oxygen-enriched air, and desorbing the non-oxygen constituent when the pressure is purged.

### CROSS-REFERENCE TO RELATED APPLICATIONS

The present invention claims the priority of the U.S. Provisional Patent Application filed on Jul. 8, 2016 with the No. 62/359,953 and entitled, “APPARATUS AND METHOD FOR CLEANING AND OXYGEN-ENRICHING AIR”, and the contents of which are included in entirety as reference of the present invention. The present application is filed as a national phase application filed in consequence/continuation of the PCT application with serial number PCT CA2017/000168 filed on Jul. 7, 2017 with the title, “APPARATUS AND METHODS FOR CLEANING AND OXYGEN-ENRICHING AIR” and the contents of which are included entirely as reference of the present invention.

### A) TECHNICAL FIELD

This relates to devices and methods for supplying breathable air.

### B) BACKGROUND OF THE INVENTION

Air pollution presents major problems, particularly in large, heavily populated cities.

Moreover, although certain buildings may have air quality controls, residents of urban centres may spend significant time outdoors or in buildings without air quality controls. Breathing of polluted air is associated with numerous adverse health and quality of life effects. Acute and chronic health issues associated with air pollution may also pose public health problems.

It has therefore become common for individuals to wear disposable face masks. However, such masks are typically difficult to breathe through and provide limited filtering and air quality improvement. Conversely, existing oxygen delivery devices tend to be bulky, complicated and difficult to use.

### C) SUMMARY OF THE INVENTION

An example portable breathing apparatus for oxygen enrichment of breathable air, comprises: an adsorption vessel; an air compressor for intermittently pumping atmospheric air into the adsorption vessel to pressurize the adsorption vessel; a valve for selectively purging pressure from the adsorption vessel by venting the vessel to atmosphere; an adsorbent disposed within the adsorption vessel for preferentially adsorbing a non-oxygen constituent of atmospheric air when the vessel is pressurized, thereby producing oxygen-enriched air, and desorbing the non-oxygen constituent when the pressure is purged.

An example method of enriching oxygen content in breathable air, comprises: pumping atmospheric air into an adsorption chamber to pressurize the adsorption chamber; adsorbing a non-oxygen constituent from the atmospheric air to produce oxygen-enriched air; outputting oxygen-enriched air for breathing; venting the adsorption chamber to atmosphere to depressurize the adsorption chamber and desorbing the non-oxygen constituent; exhausting the non-oxygen constituent from the adsorption chamber.

An example device for producing oxygen enriched air comprises: a single renewable adsorbent chamber comprising: an inlet for receiving atmospheric air having a first flow resistance, and a one-way outlet for delivering the oxygen enriched air outside the single renewable adsorbent chamber having a second flow resistance, wherein the first flow resistance is greater than the second flow resistance; and an adsorbent with an adsorbent surface area defining a production rate of the oxygen enriched air; wherein the atmospheric air passes through the inlet and contacts the adsorbent surface area at a first chamber pressure to produce the oxygen enriched air.

An example process for producing oxygen enriched air, comprises: contacting atmospheric air with an adsorbent within a single renewable adsorbent chamber at a first pressure such that a non-oxygen constituent of atmospheric air is preferentially adsorbed to the adsorbent relative to oxygen to produce oxygen enriched air; depressurizing the single renewable adsorbent chamber to a second chamber pressure such that the adsorbed non-oxygen constituent is desorbed from the adsorbent.

Other aspects will be apparent to skilled persons from the disclosure herein.

### D) BRIEF DESCRIPTION OF THE DRAWINGS

In the drawings, which show example embodiments:

FIG. 1 is a perspective view of an individual with a breathing apparatus;

FIGS. 2A-2B are front and rear perspective views of the breathing apparatus of FIG. 1;

FIGS. 3A-3B are front and rear perspective views of the breathing apparatus of FIG. 1, with housing components omitted;

FIGS. 3C-3D are schematic cross-sectional views of example adsorption vessels;

FIG. 4 is an exploded view of an adsorption vessel of the breathing apparatus of FIG. 1;

FIG. 5 is a schematic diagram of fluid connections between components of the breathing apparatus of FIG. 1;

FIG. 6 is a schematic diagram of electrical connections between components of the breathing apparatus of FIG. 1;

FIGS. 7A-7D are schematic diagrams showing operating states of valves of the breathing apparatus of FIG. 1;

FIGS. 8A-8B are flow charts depicting methods of oxygen-enriching breathable air;

FIG. 9 is a timing diagram showing operational states of components of the breathing apparatus of FIG. 1 and oxygen content;

FIG. 10 is a schematic of another breathing apparatus;

FIG. 11 is an isometric view of another breathing apparatus, with the housing partially cut away to show internal components; and

FIG. 12 is an isometric view of another breathing apparatus, with the housing partially cut away to show internal components.

### E) DETAILED DESCRIPTION OF THE INVENTION

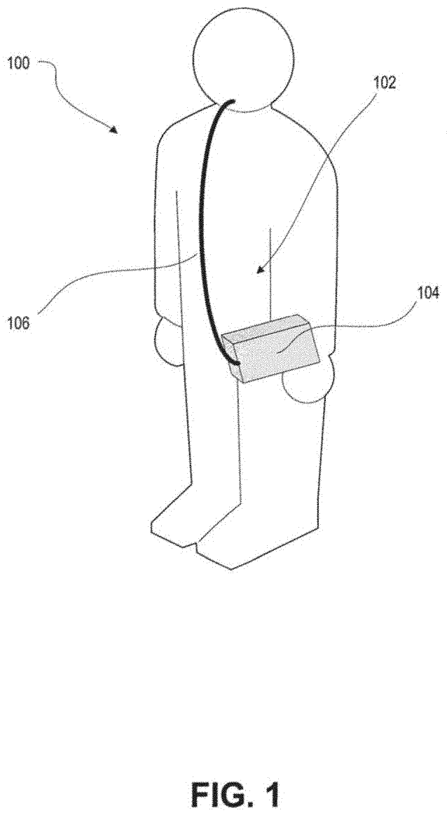

FIG. 1 depicts an individual user 100 with an example breathing apparatus 102. Breathing apparatus 102 includes an oxygen enriching unit 104 and a breathing tube 106. Breathing apparatus 102 may draw atmospheric air into oxygen enriching unit 104, increase its oxygen content, and deliver it to user 100 through breathing tube 106.

Breathing apparatus 102 may be worn by, e.g. mounted to, user 100. For example, breathing apparatus 102 may be attached to a belt or article of clothing of user 100, e.g. using a clip. Alternatively, breathing apparatus 102 may be carried in a pack.

Breathing tube 106 may be any suitable conduit for delivering breathable air to individual 100. In some embodiments, breathing tube 106 may be formed of flexible plastic or rubber for conforming to the wearer's movement. Breathing tube 106 may have a mask (not shown) suitable for engaging the nose or mouth of the wearer. In some embodiments, the mask may form a seal or substantially form a seal with the wearer's face. For example, breathing tube 106 may be configured to connect with, or may be integrally formed an appropriately modified full-face or half-face respirator mask such as a 3M 6900 Full-Face Respirator or 3M Medium Professional Multi-Purpose Respirator. In particular, the breathing tube 106 may be attached to an inhale valve. When masks are used, a flow of oxygen of 5-6 L/min may avoid CO2 accumulation within the mask, although various schemes of separating inhale and exhale are known and rebreathing may be avoided through use of one way valves to sequester expired gas from inspired gas. Additionally, the tube 106 can be used in conjunction with (e.g. connected to or integrally formed with) a nasal cannula.

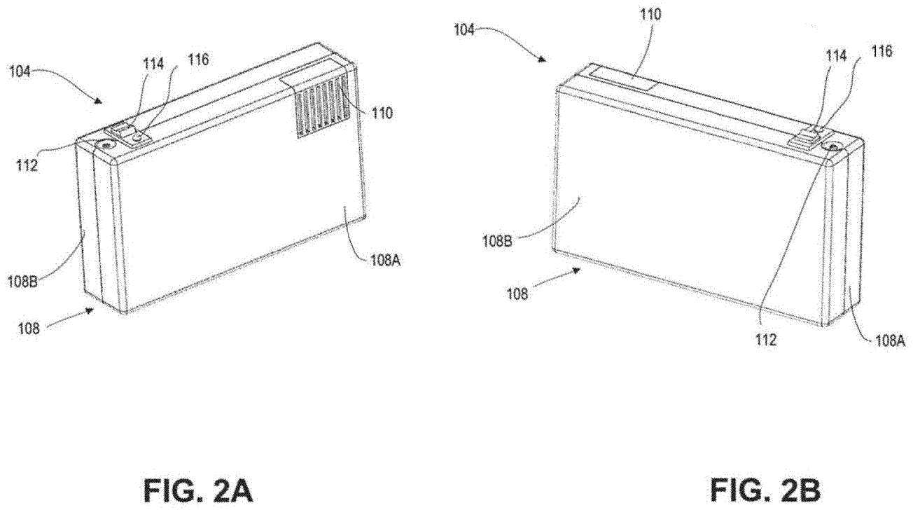

FIGS. 2A and 2B depict front and rear isometric views of oxygen enriching unit 104. As depicted, oxygen enriching unit 104 includes an outer housing 108 which may be formed in multiple pieces, e.g. front and rear covers 108A, 108B.

Oxygen enriching unit 104 includes an air box 110 through which outside air may be drawn into housing 108 and through which air may be exhausted from housing 108. Oxygen enriching unit 104 also has a breathing outlet 112 for dispensing oxygen-enriched air. As used herein, the term “oxygen-enriched air” refers to air having oxygen concentration above that of atmospheric air. Alternatively, separate air boxes may be provides for intake and exhaust. That is, air box 110 may be a first air box and may be used to allow outside air to be drawn into housing 108, while a second air box may be provides, through which air exits housing 108.

Oxygen enriching unit 104 includes controls accessible outside of housing 108. For example, oxygen enriching unit 104 includes a main power switch 114 for activating oxygen enriching unit 104. A status indicator may also be provided, such as a light-emitting diode (LED) 116. LED 116 may illuminate in one or more colours or patterns to indicate an operation Status of oxygen enriching unit 104.

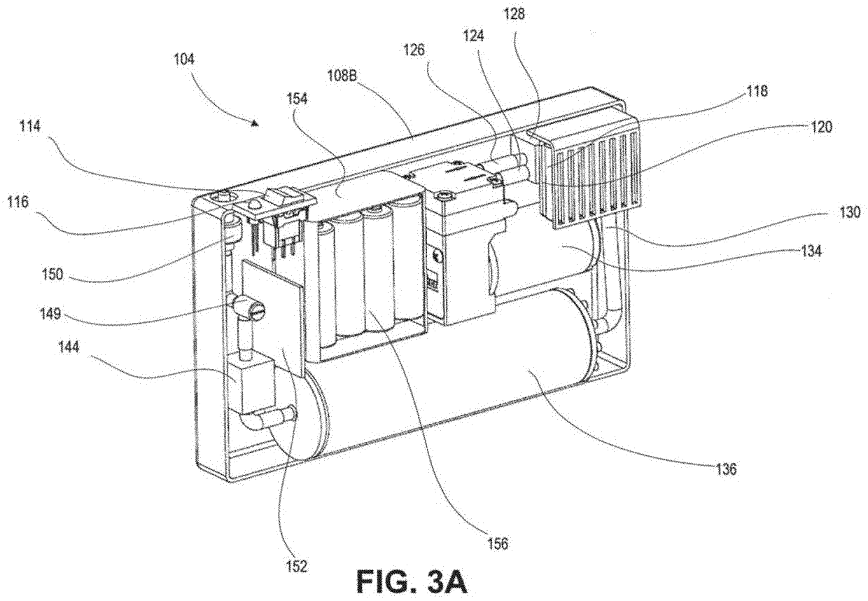

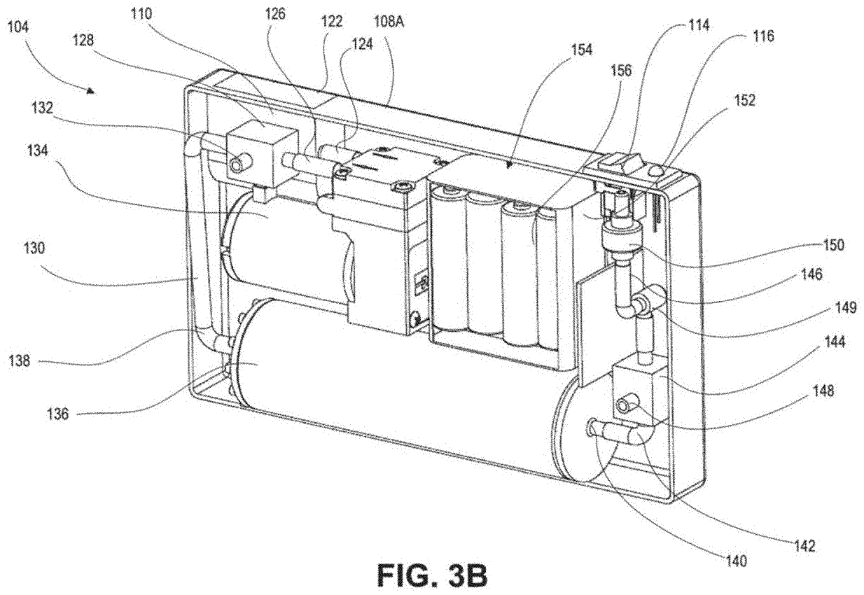

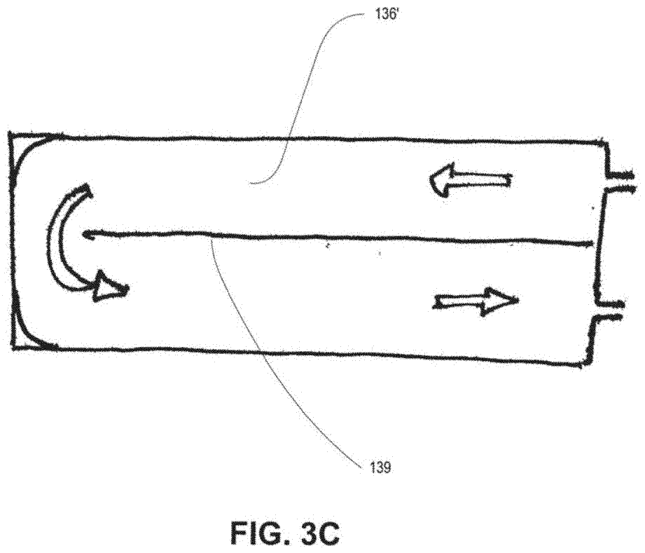

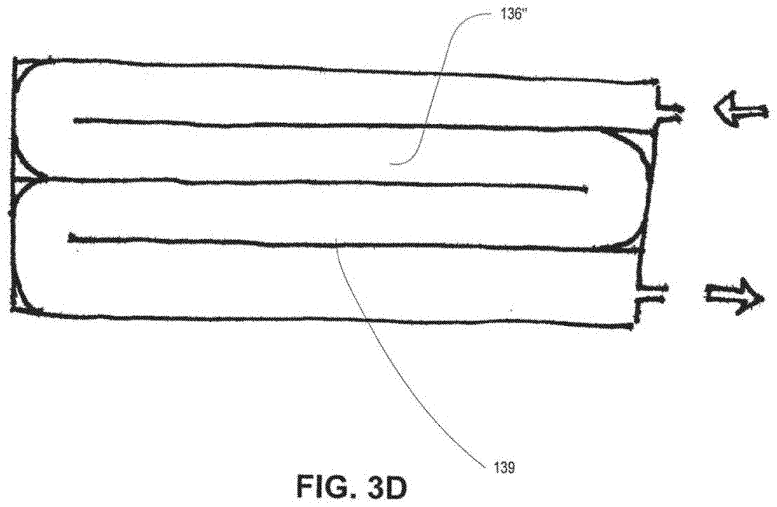

FIGS. 3A and 3B shows isometric views of oxygen enriching unit 104, with front cover 108A and rear cover 108B omitted, respectively, to show internal components.

Air box 110 of oxygen enriching unit 104 may include an intake filter 118. Intake filter 118 may include one or more filtering elements such as a grate or screen for removing large particles entrained in intake air. Intake filter 118 may further include fine filtering elements, such as paper or cloth filters. Such filters may be certified according to suitable filtering standards. For example, filter 118 may include a HEPA filter or the like. In some embodiments, intake filter 118 may be capable of removing airborne particulates; in particular, fine particulates with diameter as low as 2.5 μm-10 μm. For example, intake filter 118 may be a PM₁₀ filter, capable of removing particulates having diameter of at least 10 μm, or a PM₂₅ filter, capable of removing particulates having diameter of at least 2.5 μm. In some embodiments, an electrostatic precipitator may be provided instead of or in addition to intake filter 118. Intake filter 116 may include a membrane-type or adsorbent filter modified with surfactant molecules for removing volatile organic compounds (VOCs) from intake air, as well as particulates.

Air box 110 may also include a desiccant package 120 and may define a flow path through filter 118 and desiccant package 120. Desiccant package 120 may contain particles of a desiccant for removing moisture from intake air. The desiccant particles may be, for example, silica gel or activated alumina desiccant. In an example, the desiccant particles may be granular particles about 0.5-0.8 mm in diameter.

Air box 110 may be removably inserted in housing 108 through an opening 122 so that filter 118 or desiccant package 120 may be easily replaced. For example, after prolonged use, filter 118 may become, clogged or desiccant package 120 may become saturated with moisture, such that they lose effectiveness. Conveniently, filter 118 and desiccant package 120 may be provided in the form of cartridges or sachets for easy installation in air box 110. Air box 110 may be retained in housing 108 by any suitable mechanism. For example, air box 110 may be slidably received in tracks defined in housing 108, or may be secured by a clip.

Air box 110 may communicate with a compressor intake tube 124 and compressor output tube 126 through a valve 128. Valve 128 may also communicate with an adsorption vessel intake/exhaust runner 130 and may further include a purge outlet 132 for venting air to atmosphere.

Valve 128 may be selectively operable in a first state to connect compressor intake tube 124 with air box 110 and with a compressor 134 and to connect compressor output tube 126 with adsorption vessel intake/exhaust runner 130. Valve 128 may further be operable in a second state to connect adsorption vessel intake/exhaust runner 130 to purge outlet 132. Valve 128 may be electrically controlled. For example, valve 128 may be switched between its states by a supplied electrical signal.

Oxygen enriching unit 104 may also include a compressor 134 for drawing air through air box 110 and pressurizing the air. In particular, compressor 134 may receive a supply of air through compressor intake tube 124 and discharge pressurized air through compressor output tube 126. Compressor 134 may be driven by an electric motor, e.g. a DC electric motor, and may be controlled by an electrical signal. Compressor 134 may be capable of producing a pressure differential of at least 100 kPa and a flow rate of 2 litres per minute or higher. In some embodiments, compressor 134 may be a model C103E-13 or C134D-13 compressor, manufactured by Parker Hannifin Corporation.

As noted, compressor output tube 126 may be connected to compressor 134 to receive pressurized air therefrom, and may be selectively connected through valve 128 to adsorption vessel intake/exhaust runner 130. Adsorption vessel intake/exhaust runner 130 may in turn connect with an adsorption vessel 136.

Adsorption vessel 136 may be a substantially airtight vessel, with a first port 138 in communication with adsorption vessel intake runner 130, and a second port 140 for discharge of processed air.

Adsorption vessel 136 may contain an adsorbent for increasing oxygen concentration of air supplied by compressor 134. In some embodiments, the reactant may be particles of a zeolite.

The zeolite may be capable of increasing oxygen concentration in air by adsorption when subjected to cyclic pressurization and depressurization. For example, the zeolite may preferentially adsorb non-oxygen constituents of air when subjected to pressure higher than atmospheric pressure (e.g. 1-2 bar above atmospheric pressure), and may desorb previously-adsorbed molecules when subjected to lower pressure (e.g. equal to or less than atmospheric pressure). Thus, by cycling of pressure within adsorption vessel 136, zeolite particles may be activated to adsorb non-oxygen gases, and subsequently refreshed (i.e. adsorption capacity restored) by release of such gases.

In some examples, the zeolite may be selected for adsorption of nitrogen when pressurized and desorption of nitrogen when depressurized. Suitable zeolites may include, for example, LTA type zeolites (e.g. 5A, 4A) or faujasite type zeolites (e.g. 13X) and their modified forms, or a mixture of these zeolites. In some examples, the zeolite may be JLOX-101 zeolite from Jianlong Chemical or Beijing-DF-13X zeolite. Alternatively or additionally, different adsorbents may be selected for adsorption of other non-oxygen constituents of air, such as carbon dioxide and VOCs.

In some embodiments, vessel 136 may be configured as a molecular sieve adsorption column. Vessel 136 may be partially or completely packed with zeolite particles. In some examples, zeolite particles may be 0.4-1.15Φ in size. The zeolite column may be loosely packed within vessel 136. In some examples, the column may be generally cylindrical and may be approximately 4-4.5 inches in length and 1.25 inches in diameter. The zeolite particles may provide surface area of approximately 100-600 m² per gram. It has been experimentally determined that, if cycled to between atmospheric pressure and a pressure approximately 2 bar above atmospheric according to methods disclosed herein, a zeolite column having such characteristics can provide up to approximately 2 litres/min of output air with oxygen concentration of up to approximately 30-50% by volume.

In some embodiments, zeolite particles may be supported by a structure within vessel 136. For example, vessel 136 may have a frame or one or more baffles supporting zeolite particles. Such vessels may define a labyrinthine flow path, packed with zeolite particles. Air may be routed through the labyrinthine path and over the zeolite particles.

FIGS. 3C-3D depict simplified longitudinal cross-sectional views of two example vessels 136′ and 136″, respectively. Vessel 136′ has one internal baffle 139. Air may flow into vessel and reverse direction around baffle 139 before flowing out of vessel 136′. Vessel 136″ has three internal baffles 139. Air may flow into vessel 136″ and reverse direction three times, i.e. around each one of the baffles 139, before flowing out of vessel 136″. Vessels 136′, 136″ may be packed with zeolite particles of type and quantity substantially as described above with reference to vessel 136.

Baffles 139 may provide for a longer flow path through the adsorption vessel, and therefore, a longer residence time of air in the adsorbent bed. This may, in turn, provide more time for the adsorbent bed to remove non-oxygen constituents. In other words, the labyrinthine path defined by baffles 139 may increase the oxygen-enriching effect of the adsorbent bed. The length of the labyrinthine flow path and thus, the residence time of air, may increase with the number of baffles. In addition to increasing residence time, the labyrinthine path defined by baffles 239 may ensure that a large fraction of the adsorbent particles are exposed to air.

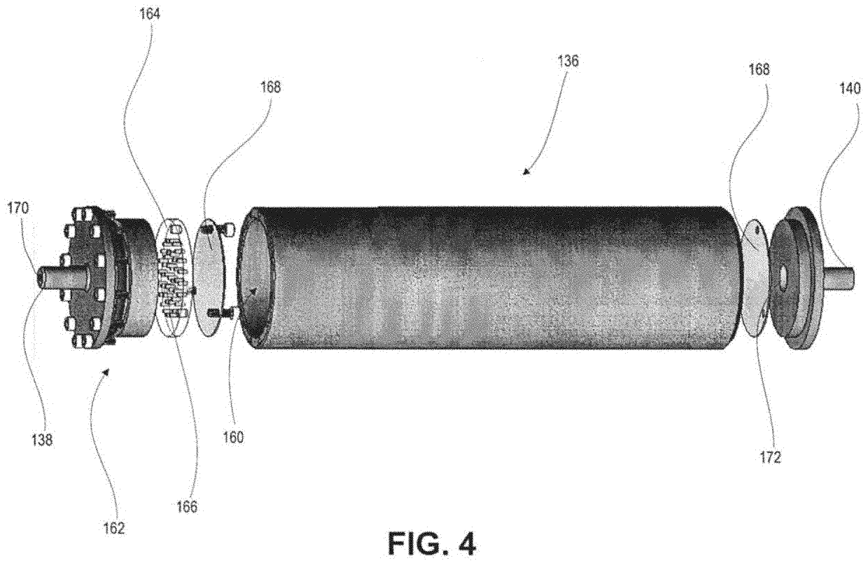

FIG. 4 depicts an exploded view of an example adsorption vessel 136 showing its internal components in greater detail.

As depicted, adsorption vessel 136 is generally hollow, defining an internal chamber 160 for cyclic pressurization and depressurization. Adsorption vessel 136 has a distributor assembly 162 at its inlet end. Distributor assembly 162 includes a plate 164 with a plurality of perforations 166. Air received through port 138 is directed against plate 164 and passes through perforations 166 to spread the inflowing air within chamber 160.

Distributor assembly 162 may define a void between first port 138 and plate 164. In some embodiments, a desiccant material may be placed therein. The desiccant material may be, for example, granular activated alumina or silica gel desiccant, and may be inserted loosely in the void defined by distributor assembly 162, or contained within a package placed therein. Atmospheric air drawn into enriching unit 104 may thus flow over the desiccant, which may remove moisture from the air. It has been determined that such removal of moisture may improve adsorption by at least some zeolites.

Adsorption vessel 136 may also include filtering elements 168 positioned proximate distributor assembly 162 and proximate second port 140. Filtering elements 168 prevent migration of desiccant or zeolite particles into or out of chamber 160. Filtering elements may be, for example, wire mesh screens or porous paper or fabric filters.

First port 138 and second port 140 of adsorption vessel 136 have respective apertures 170, 172 for flow of air into and out of vessel 136. In some embodiments, apertures 170, 172 may be configured to avoid introducing back pressure in vessel 136. That is, aperture 172 may be less restrictive (e.g. larger) than aperture 170 such that port 140 restricts air flowing out of vessel 136 no more than port 138 restricts air flowing into vessel 136.

Port 140 of vessel 136 leads to a primary vessel output tube 142, which communicates with a valve 144. Valve 144 is operable in a first state to connect primary vessel output tube 142 to a secondary vessel output tube 146 and in a second state to connect primary vessel output tube 142 to a vent tube 148 for venting vessel 136 to atmosphere. Valve 144 may be electrically controlled. That is, valve 144 may be toggled between its states by an electrical signal.

Secondary vessel output tube 146 leads to breathing outlet 112 by way of a one-way check valve 150. Check valve 150 is configured to freely allow air to flow out of oxygen enriching unit 104 through breathing outlet 112, and to prevent air from flowing into oxygen enriching unit 104 through outlet 112.

A flow control valve 149 may be placed between valve 144 and check valve 150. Flow control valve 149 may be, for example, a needle valve. Flow control valve 149 may be capable of opening a variable amount and may be electronically controlled to permit air to flow through at a specific flow rate. White valve 144 is in its first state, i.e. while vessel 136 is in communication with breathing outlet 112, valve 149 may be opened such that it permits airflow to the breathing outlet, but restricts the flow to maintain elevated pressure within chamber 136. This may in turn prevent zeolite within chamber 136 from releasing non-oxygen constituents, thereby maintaining oxygen enrichment of the air delivered through breathing outlet 112.

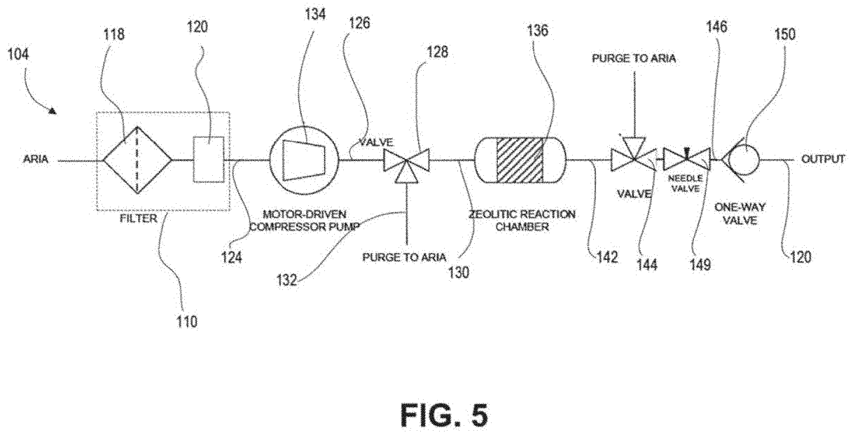

FIG. 5 shows a schematic diagram of fluid connections between components of oxygen enriching unit 104.

As depicted, air is drawn from atmosphere through airbox 110, thus passing through filter 118 and desiccant 120. Intake air is provided to compressor 134 via compressor inlet tube 124. Compressed air is supplied from compressor 134 to vessel 136 by way of compressor output tube 126, valve 128 and vessel inlet/outlet runner 130.

As noted, valve 128 may instead open to allow venting of pressure from within vessel 136 to atmosphere through vent tube 132.

Vessel 136 communicates with valve 144 through primary vessel outlet tube 142. Valve 144 connects primary outlet tube 142 to secondary outlet tube 146. One-way check valve 150 is interposed between secondary outlet tube 146 and outlet port 120. Flow control valve 149 is interposed between valve 144 and check valve 150.

Referring to FIG. 3B, oxygen enriching unit 104 further includes a controller module 152 and a power supply 154, each of which may be electrically connected with compressor 134 and each of valves 128, 144 by suitable wiring (not shown).

Power supply 154 may, for example, include one or more batteries 156, which may be connected in parallel or in series depending on the voltage and current requirements of compressor 154 and valves 128, 144. Although power supply is shown as battery powered, the power supply may include an AC/DC converter and may supply power to the unit or for recharging rechargeable batteries from a wall socket. The power supply may also receive power from cables with USB, micro USB, lightning or firewire connectors, and the like. The power supply may also receive power wirelessly using inductance or other known mechanism.

Controller module 152 may include one or more controller chips or printed circuit boards, and may be operable to provide signals to each of compressor 134 and valves 128, 144 to activate the compressor 134 or valves 128 at desired times or for a desired period of time. In some embodiments, controller module 152 may additionally have inputs for receiving signals indicative of the state of components of oxygen enriching unit 104. For example, controller module 152 may have an input for receiving a signal indicative of pressure in vessel 136 or oxygen content of air exiting vessel 136. Controller module 152 may also contain a processor and wireless data circuitry for communicating data signals with external equipment such as a smartphone or calibration instrument. In some embodiments, controller module 152 may communicate with an oxygen sensor positioned near breathing outlet 112. The oxygen sensor may provide a signal to controller module 152 indicative of the oxygen content of air discharged through breathing outlet 112. The signal from the oxygen sensor may be used, for example, to test the operation of oxygen enriching unit 104. Test results may be provided by way of a display element such as a screen, one or more LED indicators or the like, or by way of wireless communication with a device such as a computer or smart phone. In some embodiments, controls may be provided to allow users to adjust the desired oxygen content of air discharged through breathing outlet 112. Controls, such as one or more dials or keypads, may be provided on oxygen enriching unit 114. Alternatively or additionally, oxygen enriching unit 104 may receive control inputs by way of wireless communication with a smartphone, computer or the like.

Controller 152 may also communicate with a pressure sensor within vessel 136 and with a moisture sensor positioned to measure moisture content of air entering vessel 136. Controller 152 may use signals from the pressure and moisture sensors to produce an output indicative of an operating state. The output may for example be presented using a display element or by way of wireless communication with a device such as a computer or smart phone. For example, controller 152 may operate LED 116 in a first colour or illumination pattern when pressure and moisture content are within design parameters, and in different colours or illumination patters to indicate that pressure or moisture content are too low or too high.

Controller 152 may include one or more timers for tracking usage time of components. For example, a timer may be provided to measure the time elapsed since an air filter, desiccant package or zeolite has been replaced. Controller 152 may output a signal Indicative of whether a component needs replacement, e.g., when the respective timer has surpassed a threshold value. The signal may be presented using a display element such as a screen or LED or by wireless communication with a devices such as a computer or smart phone.

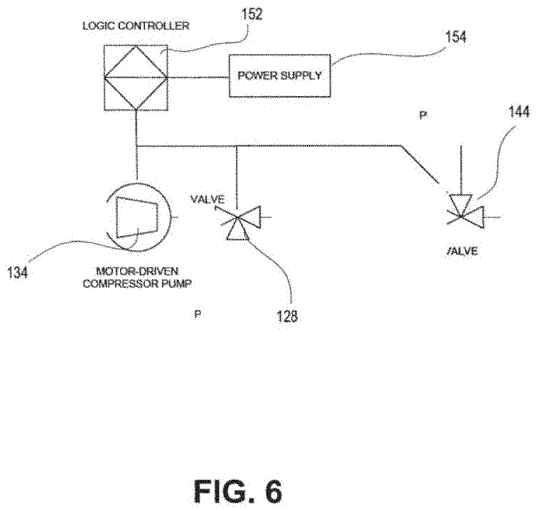

FIG. 6 is a schematic diagram of electrical connections between components of oxygen enriching unit 104. As depicted, power supply 154 is connected to controller module 152 and provides power to compressor 134, valve 128 and valve 144 through controller module 152. Controller module 152 may be configured to deliver power to compressor 134, valve 128 and valve 144 at specific time intervals, so that compressor 134 runs periodically for a specific duration, and so that valves 128, 144 switch states periodically at specific times. As will be described in further detail below, such operation may allow for pressurization of vessel 136 for effective adsorption of non-oxygen molecules from intake air, and subsequent depressunzation a vessel 136 for desorption and exhaust of the non-oxygen molecules. Pressurization and depressurization of vessel 136 may be cyclical, and the duration of each stage may be controlled to produce a specific desired oxygen concentration in output air and to provide for refreshing of zeolite between cycles.

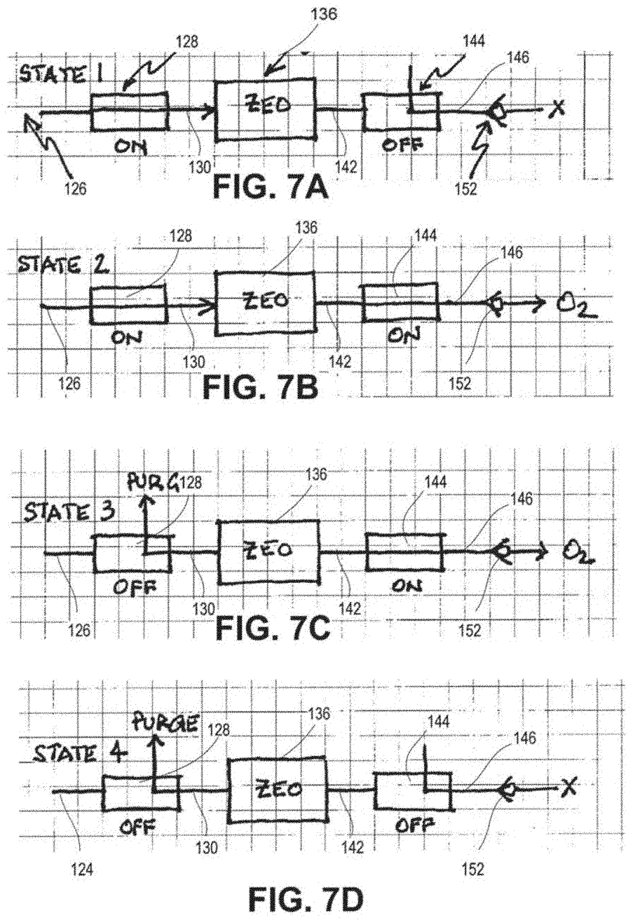

FIGS. 7A-7D depict four operating states of valves 128, 144. In a first operating state, depicted in FIG. 7A, valve 128 connects compressor output tube 126 with reactor input/exhaust runner 130. Pressurized air from compressor 134 is supplied to vessel 136. Valve 144 blocks primary vessel outlet tube 142 and vents secondary vessel outlet tube 146 to atmosphere. Accordingly, in this state, compressed air may be forced into vessel 136 and may be prevented from flowing out of vessel 136.

In a second state, depicted in FIG. 7B, valve 128 connects compressor output tube 126 with reactor input/exhaust runner 130. Valve 144 connects primary vessel outlet tube 142 with secondary vessel outlet tube 146. Accordingly, in this state, compressed air may be forced into vessel 136 and flowing out of vessel 136 and through primary and secondary outlet tubes 142, 146 to breathing outlet 112.

In a third state, depicted in FIG. 7C, valve 128 vents reactor input/exhaust runner 130 to atmosphere and blocks compressor output tube 126. Valve 144 connects primary vessel outlet tube 142 with secondary vessel outlet tube 146. Accordingly, in this state, air may be vented from vessel 136 through valve 128 and may be flow from vessel 136 through primary and secondary outlet tubes 142, 146 to breathing outlet 112.

In a fourth state, depicted in FIG. 7D, valve 128 vents reactor input/exhaust runner 130 to atmosphere and blocks compressor output tube 126. Valve 144 block primary vessel outlet tube 142 and vents secondary vessel outlet tube 146 to atmosphere. Accordingly, in this state, air may be vented from vessel 136 through valve 128.

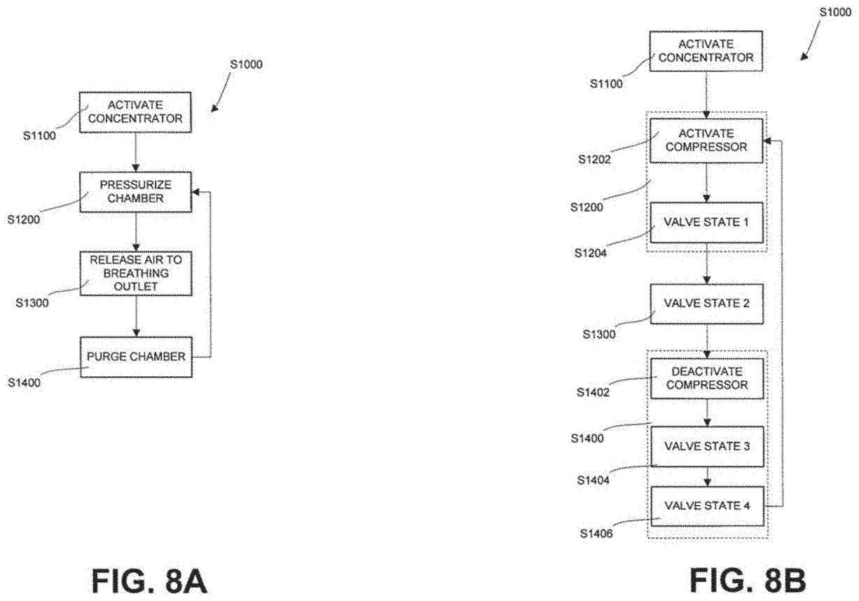

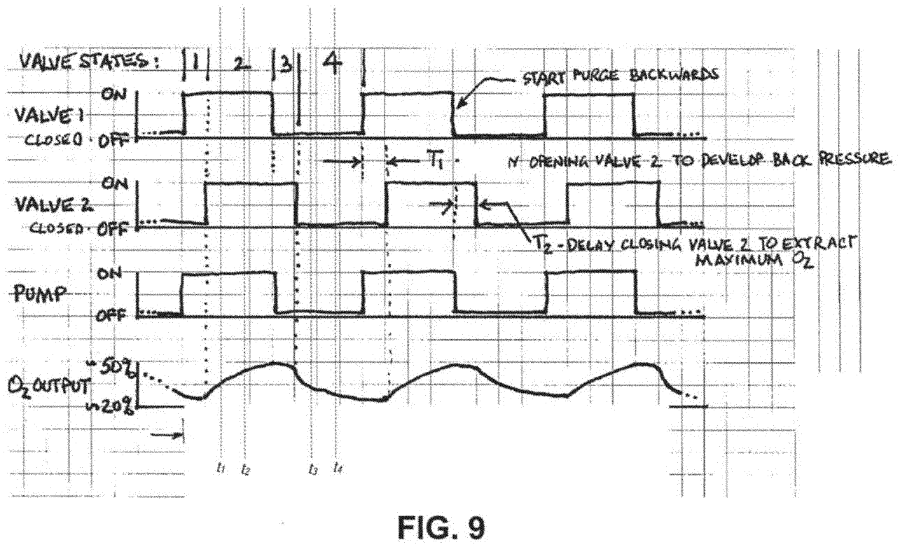

FIGS. 8A-8B depict a method S1000 of operation of oxygen enriching unit 104. FIG. 8A depicts a sequence of operational stages and FIG. 8B depicts corresponding changes of states of valves 128, 144 and compressor 134. FIG. 9 is a timing diagram depicting corresponding states of valves 128, 144 and compressor 134, along with an example plot of oxygen content of air in vessel 160.

At block S1100, oxygen enriching unit 104 is activated by a user, for example, by operation of power switch 114.

At block S1200, oxygen enriching unit 104 pressurizes chamber 160 of adsorption vessel 136. Specifically, at block S1202 (time t₁ in FIG. 9), controller module 152 sends a signal to compressor 134, causing activation of the compressor so that it draws in air through intake tube 124 and outputs pressurized air through output tube 126.

At block S1204 (time t₁ in FIG. 9), controller module 152 further sends a signal to valve 128, causing valve 128 to connect compressor output tube 126 with vessel intake/exhaust runner 130. Controller module 152 further sends a signal to valve 144, causing valve 144 to block primary vessel output tube 142 and vent secondary vessel output tube 144 to atmosphere. Thus, valves 128, 144 are placed in the first state depicted in FIG. 7A. Controller module 152 may begin a timer upon activation of compressor 134.

With compressor 134 running, and valves 128, 144 in the first state depicted in FIG. 7A, air is forced into vessel 136, causing pressure within chamber 160 to build. Controller module 152 may continue running compressor 134 for a specific time interval, i.e. from t₁ to t₂ in FIG. 9 or until a certain pressure threshold has been reached, or until a pressure above the threshold has been maintained for a period of time.

The pressure reached within chamber 160 may depend on characteristics of compressor 134. In some embodiments, compressor 134 may be capable of producing a pressure increase of at least 2 bar, and a flow rate of approximately 2 litres per minute. In such examples, pressure within chamber 160 may reach approximately 2 bar above atmospheric pressure.

Adsorption of nitrogen by zeolite within chamber 160, and the rate of such adsorption may depend on pressure within chamber 160, the type, amount and saturation of the zeolite, and residence time of air within chamber 160, among other factors. The duration for which compressor 134 is run with valve 144 closed may be selected according to the desired oxygen content in output air in view of such factors, as well as the pressure increase and flow rate produced by the compressor, and the volume of vessel 136 and chamber 160.

With a compressor 134, vessel 136 and zeolite as described herein, at block S1100, compressor 134 may be operated at a maximum pressure increase of approximately 2 bar for approximately 10 seconds, which may produce sufficient pressure in chamber 160 to cause nitrogen adsorption.

At block S1300 (time t₃ in FIG. 9), controller module 152 sends a signal to valve 144, causing valve 144 to connect primary outlet tube 142 with secondary outlet tube 146, allowing air to flow to flow control valve 149. Controller module 152 likewise sends a signal to valve 149, causing valve 149 to open sufficiently to permit airflow and maintain pressure in chamber 136. Air flows from chamber 136, by way of valves 144, 149, 150, through breathing outlet 112 to the user. Thus, at block S1300, valves 128, 144 are placed in the second state, depicted in FIG. 7B. At block S1300, controller module continues to send a signal to compressor 134 causing operation of the compressor.

When valves 128, 144 are transitioned to their second state at block S1300, pressure within vessel 136 is elevated. Therefore, upon opening of valve 144, a pressure differential exists across valve 144 and air is urged out of vessel 136 toward breathing outlet 112.

Air forced into vessel 136 may reside within chamber 160 for a period of time until it is forced or drawn out through outlet tubes 142, 146 and breathing outlet 112. During its residence in chamber 160 under elevated pressure, nitrogen may be adsorbed by zeolite in chamber 160. Accordingly, air exiting vessel 136 may be higher in oxygen content than atmospheric air. In some examples, oxygen content in air exiting vessel 136 may be as high as 50% by volume.

As more nitrogen is adsorbed by zeolite in chamber 160, the rate of adsorption may slow down. Accordingly, at block S1400 (time t₄ in FIG. 9), controller module 152 may cause purging of vessel 136. Specifically, at block S1402, controller module 152 causes compressor 134 to stop running, for example by sending a signal to compressor 134 or by interrupting power to compressor 134.

At block S1404, controller module 152 sends a signal to valve 128 causing valve 128 to block compressor output tube 126 and vent vessel inlet/exhaust tube 130 to atmosphere. Thus, at block S1404, valves 128, 144 are placed in the third state, depicted in FIG. 7C.

While valves 128, 144 are in the third state, depicted in FIG. 7C, air may freely flow from chamber 160 to atmosphere through inlet/exhaust tube 130 and valve 128. Accordingly, pressure in chamber 160 may be released. As valve 144 remains open, a user may continue to breathe air through breathing outlet 112.

At block S1406, controller module 152 sends a signal to valve 144, causing valve 144 to block primary outlet tube 142 and vent secondary outlet tube 146 to atmosphere. Thus, at block S1406, valves 128, 144 are placed in the fourth state depicted in FIG. 7D. In this state, pressure in vessel 136 may continue to be vented through valve 128. Meanwhile, compressor 134 remains inactive.

With valve 128 open for venting, conditions within vessel 136 (e.g. pressure, oxygen and nitrogen content) may approach or equalize with atmospheric conditions. Depressurization of chamber 160 may cause nitrogen desorption by the zeolite. As nitrogen is desorbed, the zeolite regains capacity to adsorb nitrogen under pressure. Purging of vessel 136 (and consequent desorption of nitrogen) may be allowed to continue until the zeolite regains substantially all of its capacity to adsorb nitrogen, so that enriching unit 104 may be cycled without degradation of its oxygen-enriching effectiveness. In examples, at block S1404, compressor 134 may remain inactive and valves 128, 144 may remain in the third state of FIG. 7C for approximately 10-15 seconds and at block S1406, compressor 134 may remain inactive and valves 128, 144 may remain in the fourth state of FIG. 7D for approximately 10-15 seconds.

The rate at which pressure is vented from vessel 136 may depend in part on the length and diameter of tubing in oxygen enriching unit 104. In the depicted embodiment, tubing within oxygen enriching unit 104 has an internal diameter of approximately 0.2 inches, and has a total length of approximately 5-6 inches. In other embodiments, tubing may be sized differently. If tubing internal diameter is substantially larger or if tubing is substantially shorter, the required venting time may be shorter (e.g., 1-2 seconds shorter). Conversely, if tubing internal diameter is substantially smaller or if tubing is substantially longer, the required venting time may be longer (e.g. 1-2 seconds longer).

In some embodiments, block S1406 may be omitted. That is, rather than closing valve 144 to close, both valves 128 and 144 may remain open throughout venting of vessel 136, so that the vessel 136 vents both through inlet/exhaust tube 130/valve 128 and through outlet tubes 142, 146/valve 144. In such cases, vessel 136 may be vented through valves 128, 144 for a total of approximately 20-30 seconds. Air vented through valve 144 may be delivered to the user for breathing, or may be vented to atmosphere.

In other embodiments, valve 128 may remain closed at block S1404. That is, rather than operating in the third state of FIG. 7C, oxygen enriching unit 104 may vent vessel 136 only through valve 144 for breathing by the user. Such venting may avoid releasing oxygen-enriched air from vessel 136 to atmosphere. In such scenarios, venting may be relatively slow, compared to the configuration of FIG. 7C.

The timing at which valves 128, 144 open and close may be configured to avoid release of air to the user with less than atmospheric oxygen concentration.

Breathing apparatus 102 and method S1000 may permit portable and economical production of oxygen-enriched air for breathing by a user. Breathing apparatus 102 may operate with at least a single adsorption chamber and at least a single compressor. Accordingly, breathing apparatus 102 may be relatively lightweight for ease of portability. Moreover, the pipe and valve configuration disclosed herein is relatively simple and control of the various components may likewise be relatively simple. This may provide for ease of operation and may allow for economical production and operation.

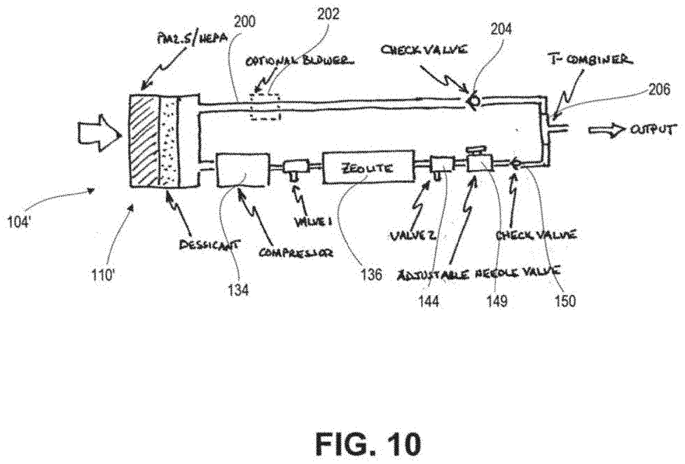

In some embodiments, oxygen-enriched air discharged from vessel 136 may be blended with other breathable air and a mixture thereof may be delivered to a user. FIG. 10 depicts a enriching unit 104′ representative of such embodiments. Oxygen enriching unit 104′ has many components similar to those of oxygen enriching unit 104, which are labelled with like reference numerals. Oxygen enriching unit 104′ has an air box 110′ which includes a filter and desiccant (e.g. a desiccant package). The air box has two outlets. One outlet leads to vessel 136 by way of compressor 134. The other outlet leads to a bypass tube 200. The bypass tube 200 provides a flow of filtered, clean breathable air to a user, without that flow passing through vessel 136. The bypass tube 200 may optionally have a blower 202 for forcing airflow there through. In addition, a check valve 204 may be positioned in bypass tube 204 for preventing reversal of airflow toward air box 110′. Bypass tube 200 and the output tube of check valve 150 meet at a T-junction 206. T-junction receives clean, breathable air that has been oxygen-enriched in vessel 136 and clean, breathable air that has bypassed vessel 136, and allows mixing of the two prior to delivery to a user. Optionally, one or more adjustable valves may be provided for operation by the user or by controller 152 to alter the ratio in which the airstreams are mixed, and thus the volume and oxygen content of air provided to the user.

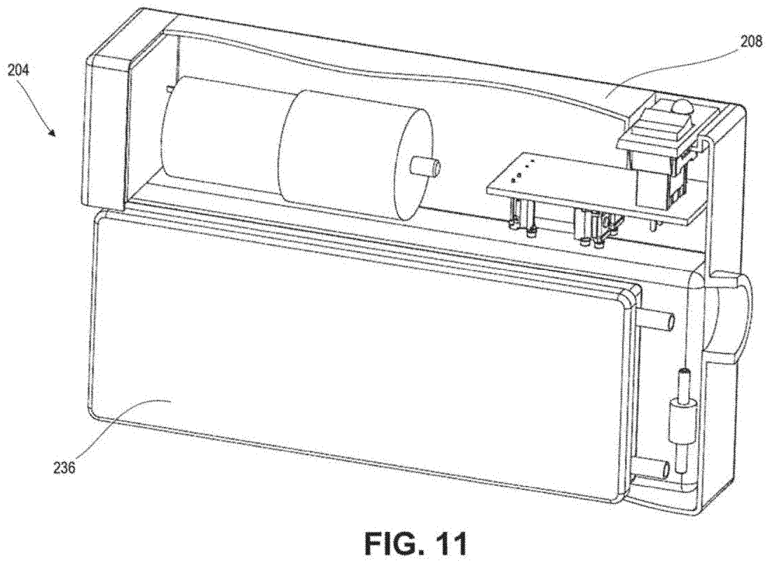

As described above, vessel 136 is generally cylindrical in shape. In other embodiments, vessel 136 may be shaped differently. For example, FIG. 11 depicts an oxygen enriching unit 204 with an elongate rectangular vessel 236, which may be referred to as a flat pack. Other components of oxygen enriching unit 204 are generally similar to those of oxygen enriching unit 104 and omitted for simplicity.

Vessel 236 may have reduced thickness relative to a cylindrical vessel 136. Likewise, housing 208 of oxygen enriching unit 204 may be thinner than housing 108 of oxygen enriching unit 104. Such reduced thickness may provide for increased portability or perception of portability by users.

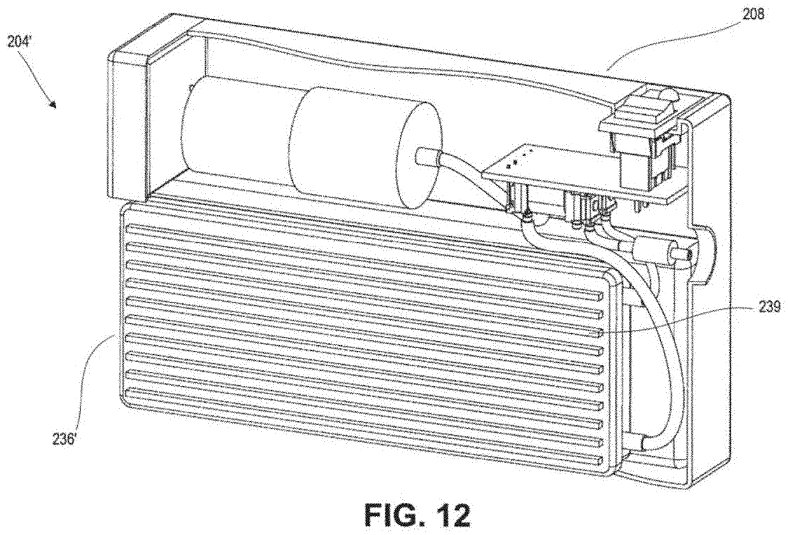

In some embodiments, the adsorbent vessel 136/236 is equipped with a series of external fins. For example, FIG. 12 depicts an oxygen enriching unit 204′, generally identical to oxygen enriching unit 204, except that it has an adsorbent vessel 236′ with fins 239. Fins 239 project outwardly from the vessel's exterior wall. Fins 239 promote dissipation of heat generated by pressurization of air within the adsorbent vessel. Dissipation of heat by the adsorbent vessel and fins 239 may be sufficiently high that the vessel can be cooled entirely passively, that is, without need for components such as cooling fans. For example, in some embodiments as described herein, with peak pressure values of approximately 2 bar (25-30 psi), cooling may be entirely passive. Notably, operating temperatures slightly above typical ambient room temperature may help eliminate moisture or humidity within the adsorbent chamber, which may in turn promote the adsorption of non-oxygen air constituents by the adsorbent particles.

Fins 239 also provide structural reinforcement of the adsorbent vessel. That is, a vessel equipped with tins 239 will generally be stiffer and stronger than an equivalent vessel (e.g. a vessel of the same size and specification) without fins 239.

Adsorbent vessels may be formed of a range of materials. In some embodiments, adsorbent vessels may be metallic, e.g. steel or aluminum. In some other embodiments, adsorbent vessels may be formed of polymers e.g., plastics. The thermal and structural effects of fins 239 may be of particular importance in embodiments with non-metallic adsorbent vessels. Specifically, in some embodiments, fins 239 provide sufficient strength or thermal dissipation for polymer vessels, while metallic vessels, such as steel or aluminum vessels, may be sufficiently strong and dissipate sufficient heat without fins 239.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface. The processor may store in the memory times of use, total duration of use since a component of the system was changed or replaced, sensor readings, sensor readings over time which constitute trends, and other data relevant to the operation of the device.

The preceding discussion provides many example embodiments. Although each embodiment represents a single combination of inventive elements, other examples may include all possible combinations of the disclosed elements. Thus if one embodiment comprises elements A, B, and C, and a second embodiment comprises elements B and D, other remaining combinations of A, B, C, D, may also be used.

The term “connected” or “coupled to” may include both direct coupling (in which two elements that are coupled to each other contact each other) and indirect coupling (in which at least one additional element is located between the two elements).

Although the embodiments have been described in detail, it should be understood that various changes, substitutions and alterations can be made herein without departing from the scope as defined by the appended claims.

We claim:

1. A portable breathing apparatus for oxygen enrichment of breathable air, comprising:
an adsorption vessel;
an air compressor for intermittently pumping atmospheric air into said adsorption vessel to pressurize said adsorption vessel;
a valve for selectively purging pressure from said adsorption vessel by venting said vessel to atmosphere;
an adsorbent disposed within said adsorption vessel for preferentially adsorbing a non-oxygen constituent of atmospheric air when said vessel is pressurized, thereby producing oxygen-enriched air, and desorbing said non-oxygen constituent when said pressure is purged.

2. The portable breathing apparatus of claim 1, wherein said adsorbent comprises a plurality of adsorbent particles defining an adsorbent surface area.

3. The portable breathing apparatus of claim 2, wherein said adsorbent comprises a zeolite.

4. The portable breathing apparatus of claim 3, wherein said non-oxygen constituent comprises nitrogen.

5. The portable breathing apparatus of claim 3, wherein said adsorbent comprises a LTA or faujasite-type zeolite.

6. The portable breathing apparatus of claim 1, wherein said air compressor is configured to pressurize said adsorption vessel to about 2 bar above atmospheric pressure.

7. The portable breathing apparatus of claim 1, wherein said adsorption vessel communicates with said valve through an exhaust outlet, and wherein said adsorption vessel has a breathing outlet for delivering said breathable air to a user.

8. The device of claim 5, wherein the LTA or faujasite-type zeolite comprises particles each having a size range of about φ0.4 mm to about φ2.5 mm.

9. The device of claim 8, wherein the LTA or faujasite-type zeolite comprises particles each having a size range of about φ0.4 mm to about φ0.8 mm.

10. The device of claim 5, wherein the LTA or faujasite-type zeolite comprises particles each having a size range of about φ0.85 mm to about φ1.15 mm.

11. The device of claim 1, wherein the single renewable adsorbent chamber further comprises a diffuser for distributing the atmospheric air received by the inlet over the reactive surface area.

12. The device of claim 1, further comprising at least one particulate filter for filtering atmospheric air.

13. The device of claim 1, further comprising a desiccant for removing moisture from said atmospheric air.

14. The device of claim 1, further comprising a plurality of cooling fins projecting from an external surface of said adsorption vessel.

15. The device of claim 1, wherein said adsorption vessel is elongate and rectangular in shape.

16. The device of claim 1, wherein said adsorbent is operable to produce air with a concentration of about 30-50% oxygen by volume, at a rate of 2 litres per minute.

17. A method of enriching oxygen content in breathable air, comprising:
pumping atmospheric air into an adsorption chamber to pressurize said adsorption chamber;
adsorbing a non-oxygen constituent from said atmospheric air to produce oxygen-enriched air;
outputting oxygen-enriched air for breathing;
venting said adsorption chamber to atmosphere to depressurize said adsorption chamber and desorbing said non-oxygen constituent;
exhausting said non-oxygen constituent from said adsorption chamber.

18. The method of claim 17, comprising pumping atmospheric air into an adsorption chamber to pressurize said adsorption chamber to a pressure about 2 bar above atmospheric pressure.

19. The method of claim 17, wherein said non-oxygen constituent comprises nitrogen.

20. The method of claim 19, wherein said adsorbing comprises adsorbing with a LTA or faujasite-type zeolite.

21. The method of claim 17, comprising diffusing said atmospheric air pumped into said chamber.

22. The method of claim 17, comprising removing moisture from said atmospheric air prior to said adsorbing.

23. The method of claim 22, wherein said removing moisture comprises flowing said atmospheric air over a desiccant.

24. A device for producing oxygen enriched air comprising:
a single renewable adsorbent chamber comprising:
an inlet for receiving atmospheric air having a first flow resistance, and a one-way outlet for delivering the oxygen enriched air outside the single renewable adsorbent chamber having a second flow resistance, wherein the first flow resistance is greater than the second flow resistance; and
an adsorbent with an adsorbent surface area defining a production rate of the oxygen enriched air;
wherein the atmospheric air passes through the inlet and contacts the adsorbent surface area at a first chamber pressure to produce the oxygen enriched air.

25. The device of claim 24, wherein the adsorbent preferentially adsorbs a non-oxygen constituent of atmospheric air at the first chamber pressure, relative to oxygen.

26. The device of claim 25, wherein the device further comprises a pressure device for cycling between the first pressure and a second pressure within said chamber.

27. The device of claim 26, wherein the first chamber is about 2 bar to about 3 bar, and the second pressure is about 1 bar.

28. The device of claim 27, wherein the adsorbed non-oxygen constituent is desorbed from the adsorbent at the second pressure.

29. The device of claim 28, wherein the non-oxygen constituent is released from the inlet after desorption.

30. The device of claim 24, wherein the absorbent is a granulated zeolite.

31. The device of claim 30, wherein the granulated zeolite is a LTA or faujasite-type zeolite.

32. The device of claim 30, wherein the granulated zeolite comprises particles each having a size range of about to φ0.4 mm to about φ2.5 mm.

33. The device of claim 30, wherein the granulated zeolite comprises particles each having a size range of about to φ0.4 mm to about φ0.8 mm.

34. The device of claim 30, wherein the granulated zeolite comprises particles each having a size range of about cp 0.85 mm to about φ1.15 mm.

35. The device of claim 24, wherein the single renewable adsorbent chamber further comprises a diffuser for distributing the atmospheric air received by the inlet over the reactive surface area.

36. The device of claim 35, wherein the diffuser is a mesh plate disposed in the single renewable adsorbent chamber.

37. The device of claim 24, wherein the device further comprises at least one particulate filter for filtering atmospheric air.

38. The device of claim 24, wherein the one-way outlet further comprises a venturi for entraining atmospheric air into the flow of oxygen enriched air.

39. The device of claim 24, wherein said adsorbent is operable to produce air with a concentration of about 30-50% oxygen by volume, at a rate of 2 litres per minute.

40. A process for producing oxygen enriched air, comprising
contacting atmospheric air with an adsorbent within a single renewable adsorbent chamber at a first pressure such that a non-oxygen constituent of atmospheric air is preferentially adsorbed to the adsorbent relative to oxygen to produce oxygen enriched air,
depressurizing the single renewable adsorbent chamber to a second chamber pressure such that the adsorbed non-oxygen constituent is desorbed from the adsorbent.

41. The process of claim 39, wherein the oxygen enriched air is delivered from the single renewable adsorbent chamber through a one-way outlet.

42. The process claim 39, wherein the first chamber pressure is about 2 bar to about 3 bar, and the second chamber pressure is about 1 bar.

43. The process of claim 39, wherein oxygen-enriched air having an oxygen concentration of about 30-50% by volume is produced at a rate of about 2 litres per minute.

44. The process of claim 39, wherein the non-oxygen comprises nitrogen.
